(19) United States
(12) Patent Application Publication
(10) Pub. No.: US 2008/0287749 A1
(43) Pub. Date: Nov. 20, 2008

(54) Method and Apparatus for Remote Patient Monitoring

(75) Inventor: James M. Reuter, Naples, FL (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/094,795

(22) PCT Filed: Oct. 30, 2006
(86) PCT No.: PCT/IB06/54015

§ 371 Date: May 23, 2008

(30) Foreign Application Priority Data
Nov. 23, 2005 (US) 60739737

(51) Int. Cl. A61B 5/00

(52) U.S. Cl. 600/300

(57) ABSTRACT

A method and apparatus for patient monitoring are described. The method and apparatus include gathering data from a patient medical device (104) and providing the data for analysis. Messages may be provided to the patient via a patient terminal (103).

The cost of health care continues to increase. One aspect of the cost associated with health care is labor. In particular, the costs associated with sufficiently staffing health care facilities are substantial. Furthermore, there is a shortage of qualified personnel to provide certain types of care. Accordingly, the labor costs coupled with a shortage of qualified health care providers can result in limited health care at a relatively high cost. Moreover, the limited level of care often results in the treatment of patients only when urgent care is needed. As is known, the costs associated with urgent care are significant.

In an effort to reduce the cost of health care and to provide a better level of care and associated quality of life to patients, preventive health care continues to be implemented by the health care community. In particular, health care providers strive to provide access to information to their patients so that patients with chronic conditions can take steps to avoid the need for urgent care and so the patients can enjoy their lives more fully in spite of their conditions.

One known technique useful in providing long-term and real time measurement data to clinicians is remote monitoring. Remote monitoring systems include various devices required for monitoring patient vital signs. These devices include scales for weight measurement, electrocardiogram (EKG) machines and sphygmomanometers, to name a few. When a measurement is taken, the data are communicated to the clinician site (e.g., hospital or physician's office). The data are then analyzed by a clinician. The clinician will then contact the patient by telephone to inform them of an action to be taken based on the measurement data. For example, if a patient has a renal condition and his/her blood pressure is elevated beyond a safe level, the clinician may call the patient to schedule an exam in the near future.

While the remote monitoring relieves the clinician of some labor requirements and provides real time and long term data, the burden remains with the clinician to call the patient and engage in a dialogue regarding the data. This process takes time from the rather limited time of the clinician. Moreover, the patient may be difficult to reach. As such, there may a delay in the communication of the information from the clinician. Accordingly, the referenced known methods of patient monitoring are both inefficient and sometime ineffective.

Further exacerbating the problems many known remote patient monitoring systems is erroneous measurement data. The sources of erroneous data can be faulty equipment, use by other than the patient, or improper measurement technique by the patient that the clinician cannot observe. For example, use of the remote monitoring weight scale by the patient's family members can provide inaccurate data regarding the patient. This may require the clinician to inquire as to the sudden change in the patient's weight, which is clearly an inefficient use of the clinician's valuable time. Similarly, if the scale were broken or otherwise malfunctioning, erroneous data may be sent and the clinician's time again put to poor use because of the follow-up call required under certain present systems.

There is a need, therefore, for a method and apparatus adapted to provide efficient communications between clinicians and patients that overcome at least some of the shortcomings described above.

In accordance with an example embodiment, an apparatus includes a patient terminal and a medical device adapted to garner measurements from a patient and to transmit data from the measurements. The apparatus also includes a clinician terminal adapted to receive manual inputs or audio inputs, or both, from a user. In addition, the apparatus also includes a server adapted to receive the inputs and the data. The server is operative to transfer the inputs to the patient terminal.

In accordance with another embodiment, a method includes measuring a vital sign and transmitting data from the measuring to a server. In addition, the method includes transmitting the data from the server to a clinician terminal. Based on the data, the method also includes inputting a message to the clinician terminal; and providing the message at the patient terminal.

In accordance with another example embodiment, an apparatus includes a patient terminal and a medical device. The apparatus also includes a functional indicator adapted to provide data on a status of the medical device and a server adapted to receive the data. Based on the data the apparatus is adapted to provide feedback to the patient terminal.

In accordance with yet another example embodiment, a method includes gathering data from a functional indicator of a medical device; transmitting data from the monitoring to a server; and based on the data, determining an appropriate action at the server.

As used herein, the terms ‘a’ and ‘an’ mean one or more; and the term ‘plurality’ means two or more.

The invention is best understood from the following detailed description when read with the accompanying drawing figures. It is emphasized that the various features are not necessarily drawn to scale. In fact, the dimensions may be arbitrarily increased or decreased for clarity of discussion.

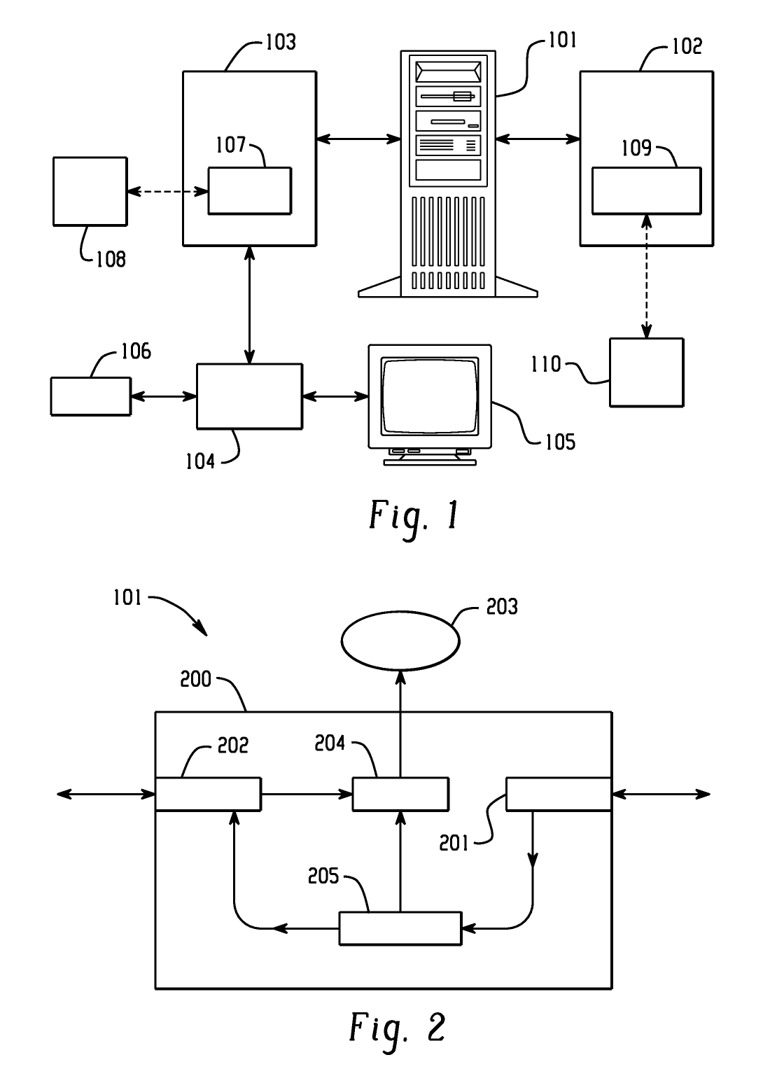

FIG. 1 is a simplified block diagram of a patient information system in accordance with an example embodiment.

FIG. 2 is a simplified block diagram of a server/central computer in accordance with an example embodiment.

FIG. 3 is a simplified block diagram of a server/central computer in accordance with another example embodiment.

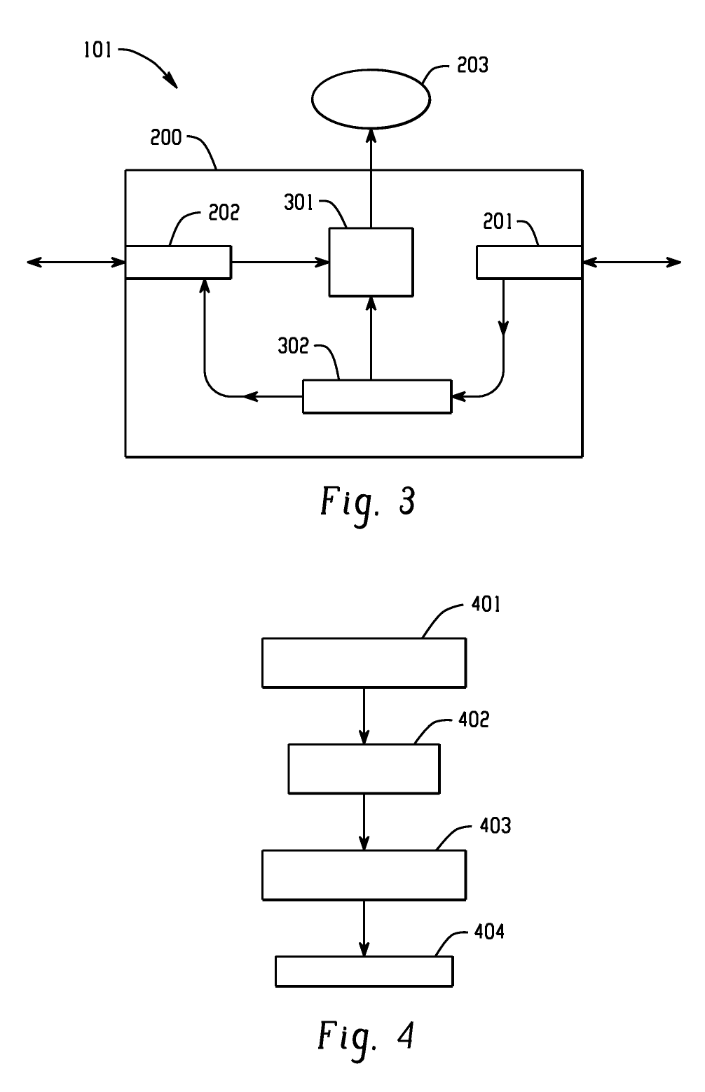

FIG. 4 is a flow-chart of a method in accordance with an example embodiment.

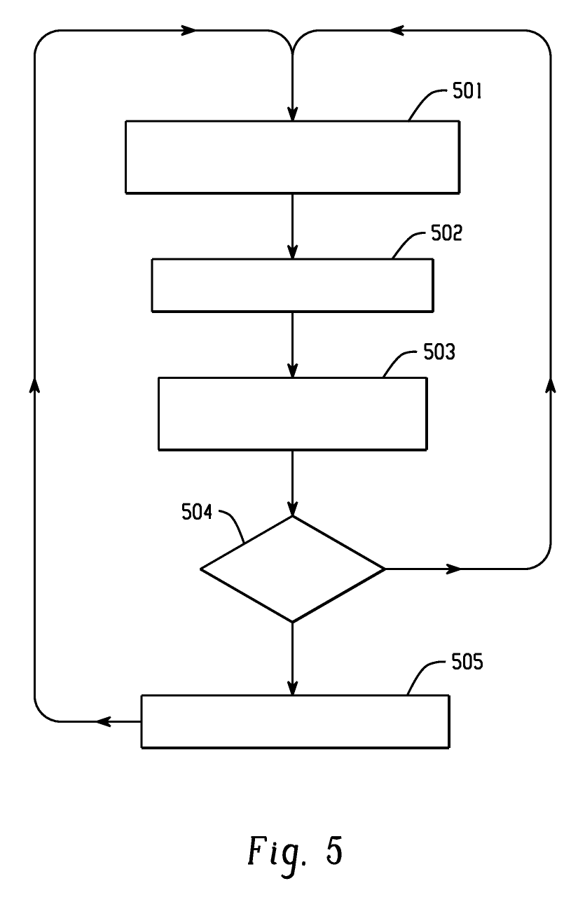

FIG. 5 is a flow-chart of a method in accordance with an example embodiment.

In the following detailed description, for purposes of explanation and not limitation, example embodiments disclosing specific details are set forth in order to provide a thorough understanding of the present teachings. However, it will be apparent to one having ordinary skill in the art having had the benefit of the present disclosure that other embodiments that depart from the specific details disclosed herein. Moreover, descriptions of well-known devices, hardware, software, methods, systems and protocols may be omitted so as to avoid obscuring the description of the example embodiments. Nonetheless, such hardware, software, devices, methods, systems and protocols that are within the purview of one of ordinary skill in the art may be used in accordance with the example embodiments. Finally, wherever practical, like reference numerals refer to like features.

FIG. 1 is a simplified block diagram of a patient information system 100 in accordance with an example embodiment. The system 100 includes a server/central computer 101, a clinician terminal 102 and a patient terminal 103. The patient terminal 103 may be in communication with a medical device 104. In an example embodiment, the medical device 104 is a device for measuring one or more patient vital signs. Illustratively, the medical device 104 may be a scale, sphygmomanometer, a hydration meter, a blood glucose meter, or a heart monitor. In addition to or instead of the medical devices noted, the medical device 104 may be a therapeutic device including but not limited to an intra-venous pump, a pacemaker, an exercise machine or an implantable cardioverter defibrillator (ICD).

The medical device 104 may be in communication with the patient terminal 103 by one of a variety of technologies. For example, for ease of use and portability about the patient's dwelling or the patient's present location, the medical device 104 may be connected via a wireless link. Such a link may include hardware and software adapted to function in accordance with one or more known wireless protocols such as IEEE 802.11 (often referred to as the WiFi standard) or IEEE 802.15 (often referred to as the Bluetooth standard), and their progeny. Accordingly, the medical device 104 and the patient terminal 103 would include the required hardware and software necessary for this communication.

Alternatively or additionally, the connection from the medical device 104 to the patient terminal 103 may be a wired connection, such as a coaxial transmission line (cable) connection. Notably, broadband communication over the cable may be via a known internet or intranet broadband protocol. The medical device 104 and the patient terminal 103 may include hardware (e.g., a modem) and software in keeping with the chosen protocol.

In a specific embodiment, the medical device 104 is a Philips Telemonitoring device commercially available from Philips Medical Systems N.A. of Bothell, Wash. USA. Notably, the Philips Telemonitoring device may be a component of the Philips M3810A Telemonitoring System also available from Philips Medical Systems, N.A. This system is described in “Philips Telemonitoring Services System Resource Guide” March 2005, the disclosure of which is specifically incorporated herein by reference. The Philips Telemonitoring device is modified in accordance with the present teachings to realize the medical device 104. Such modifications include modifications to the software and hardware of the Telemonitoring device or system, or both, to realize the medical device 104 of the specific embodiment.

The medical device 104 may include monitors 105, or sensors 106, or both. The monitors 105 and sensors 106 may be referred to herein as functional indicators and are adapted to provide a status of the function of the medical device 104. For example, a medical device 104 may operate on a direct current (DC) source such as a battery. Illustratively, the sensor 106 may be a simple voltmeter that measures the voltage of the battery. The sensor 106 may be adapted to provide the voltage reading periodically or when a threshold level is reached, or both. These readings are provided to the patient terminal 103 and to the server 101. The server 101 is adapted to compare the received voltage data and determine if action must be taken. As described herein, the server 101 may then transmit a message to the patient terminal 103 indicating that the battery level is low at a particular medical device 104 so the patient may address the problem at the medical device.

The functional indicators usefully periodically check the function of a medical device 104 to ensure that the device is properly functioning. For example, the monitoring device 106 may include self-test hardware/software adapted to test the function of a device. In a specific embodiment, the self-test hardware/software generates a test signal in an EKG device and compares the output to the test signal. The data from the self-test routine may be provided to the server 101. Based on the data, the server 101 may send a message to the patient terminal instructing the patient to take certain actions.

In an illustrative embodiment, the functional indicators 106 are adapted to gather data related to the circumstances of the measurement of vital signs data. These data qualify the measurement data. For purposes of illustration, qualifying data may comprise: signal quality (e.g., signal-to-noise-ratio (SNR)); data variance across multiple samples or measures provided in a single reading; elapsed time to acquire the vital sign data; the time of day and date the data were acquired.

The medical device 104 and the functional indicators may transmit data directly to the server 101, rather than via the patient terminal 103. This transmission and reception of data may be via one or more of the types of communication links referenced above that connect the patient terminal 103 to the server 101. Notably, the patient terminal 103 will receive messages from the server and transmit messages to the server as described.

The patient terminal 103 may be a personal computer having the requisite presentation layer software (user interface software) for interfacing with the medical device 103 and server 101. Alternatively, the patient terminal 103 may be a dedicated device such as a stand-alone terminal with a display for viewing messages and a keypad or other interface for inputting messages to the terminal 103. Notably, such a stand-alone device also includes presentation layer software for interfacing with the medical device 103 and the server 101.

In a specific embodiment, the patient terminal 103 is a Philips TeleStationO device (e.g., a TeleStation® M3812B) commercially available from Philips Medical Systems N.A. of Bothell, Wash. USA, and as described in the incorporated publication listed above. Notably, the TeleStation® is modified in accordance with the present teachings to realize the terminal 103. Such modifications include modifications to the software and/or hardware of the TeleStation® device to realize the terminal 103 of the specific embodiment.

In other specific embodiments, the patient terminal 103 may be a television (TV), or a TV with a set-top box (STB) or a personal computer (PC). The patient terminal may also be a mobile communication device such as a cellular telephone, a PDA or a portable computer. Certain modifications to the hardware, or the software, or both of the personal computer, the cellular telephone, the PDA or the portable computer may be necessary to implement the patient terminal 103 of the specific embodiments. Such modifications include modifications to the software and/or hardware to realize the terminal 103 of the example embodiment.

In still another example embodiment, the patient terminal 103 may comprise a stationary device 107 and a remote access device 108. For example, the patient terminal 103 may include a Philips Telestation device, or a PC that is fixed or stationary. The remote access device 108 is adapted to communicate with the fixed device 107.

The remote access device 108 may be a custom device with a manual interface, or an audio interface, or both, and a display. Alternatively, the remote access device 108 may be a PDA, or cellular phone or a pager. In any case, the remote access device 108 is implemented in hardware and software to communicate with the fixed device. Illustratively, this hardware and software may be in accordance with one or more of the wireless standards noted previously.

Beneficially, the remote access device 108 allows the patient or authorized person, or both, to access the data/messages from the stationary device 107 and to take appropriate action. This action may include an acknowledgement message to the server 101, or a therapeutic action based on the message received.

The patient terminal 103 may be connected to the server 101 through a wired or wireless connection well-known to one skilled in the art of information technology. For example, the connection to the server 101 may be via a plain old telephone service (POTS) line using a suitable internet protocol such as digital subscriber line (DSL) and it progeny, or via a cable-modem broadband link. Alternatively, the connection may be via one of the noted wireless protocols noted previously. Because these communication methods and apparati are known, details related thereto are not provided to avoid obscuring the description of the embodiments.

The server 101 may be a personal computer or server commercially available and modified in keeping with the present teachings. The server 101 is described in further detail in connection with FIGS. 2 and 3.

The server 101 may be resident at the clinician site (not shown) or may be a host server provided, for example, by a chosen internet service provider. The server 101 is adapted to connect one or more clinician terminals 102 with one or more patient terminals 103. Alternatively, the server 101 may be resident within one or more clinician terminals 102. For example, the clinician terminal 102 may be modified in keeping with the present teaching to include the required hardware or software, or both, to include the server 101.

In accordance with an example embodiment, the server 101 is connected to the clinician terminal 102 through a wired or wireless connection well-known to one skilled in the art of information technology. For example, the connection to the server 101 may be via a (POTS) line using a DSL line, or via a cable-modem broadband link. Alternatively, the connection may be via one of the noted wireless protocols noted previously.

The clinician terminal 102 is adapted to receive data from the patient terminal 103 and to provide messages to the patient terminal 103. These messages may be in response to data received unprompted messages to the patient such as a reminder, a message of motivational encouragement, or some other personal message.

In an example embodiment, the clinician terminal 102 is a personal computer. Alternatively, the clinician terminal 102 may be a portable device such as a portable computer, or a PDA, or a cellular telephone. Notably, each of these devices includes a manual interface, such as a key pad that allows the clinician to input a text message in response to measurement data received from the patient. The text message will then be transmitted to the patient terminal 103. The patient terminal 103 includes memory for storing the message and a display so the message may be displayed.

Alternatively or additionally, the clinician terminal 103 may be adapted to receive an audio message from the clinician. This message may be transmitted to the server 101 and to the patient terminal 103. The patient terminal 103 may include a memory so the message may be stored and an audio speaker so the patient may listen to the message. In an embodiment, the server 101 or the clinician terminal 102 are adapted to convert the audio signal from the clinician into text. The text message is then transmitted to the patient terminal 103 as described.

In another example embodiment, the clinician terminal 102 may comprise a stationary device 109 and a remote access device 110. For example, the clinician terminal 102 may include a PC that is fixed or stationary. The clinician terminal 103 may also include a remote access device 110 adapted to communicate with the fixed device 109.

The remote access device 110 may be a custom device with a manual interface, or an audio interface, or both, and a display. Alternatively, the remote access device 110 may be a PDA, or cellular phone or pager. In any case, the remote access device 110 is implemented in hardware and software to communicate with the stationary device 109. Illustratively, this hardware and software may be in accordance with one or more of the wireless standards noted previously.

Beneficially, the remote access device 110 allows the clinician to access the data/messages from the server 101 and to take appropriate action. This action may include a follow-up message, or an instruction to the patient to take a particular action.

From the description above, it will be appreciated that the patient information system 100 provides a number of different types of data for analysis by the server 101 or the clinician, or both; and these analyses may prompt a message to the patient terminal 103. First, there are the actual patient measurement data. Moreover, there are data garnered for analysis that may indicate an error in the measurement. These data may include vital sign data. For example vital sign measurements may be analyzed and the conclusion reached that the instrument is malfunctioning or is being used by other than the patient. After algorithmic computation at the server 101, a message may be sent to the patient terminal. The message may instruct the patient to check the accuracy of the device and remind the patient to prevent others from using the scale.

In addition, the patient system 100 is adapted to provide data related to the circumstances of the measurements. These data may include measurement times and days. The measurement data and the time/day data may provide a more complete analysis of the state of the patient's health. For example, if weights tend to rise during the weekend, a properly timed message might remind the patient to watch his diet and take all of his/her medicine just before each weekend.

FIG. 2 is a simplified block diagram of the server 101 in accordance with an example embodiment. Certain features of the server 101 have been described in connection with the system 100 in connection with FIG. 1. The details of these features are not repeated.

The server 101 includes a processor 200 such as a Pentium® processor commercially available from Intel Corporation, USA. The server 101 includes an interface 201 suitable for connecting the server to the clinician terminal 102. In addition, the server 101 may include another interface 202 adapted to connect the server 101 to the patient terminal 103.

Illustratively, in the event that communications between the server 101, and the clinician terminal 102 and patient terminal 103 are over an intranet connection, the respective interfaces 201, 202 may include an Ethernet network interface card (NIC). Alternatively, the server 101 may include a broadband modem for connections to the patient. Still alternatively, the interfaces 201, 202 may be POTS interfaces, wireless interfaces or fiber optic interfaces. The interfaces 201, 202 also include the requisite communications hardware (e.g., transmitter and receiver) and software to effect the transmission and reception of information between the server 101 and the clinician and patient terminals 102, 103, respectively.

The server 101 includes a database 203, such as a structured query language (SQL) database that includes data garnered from the patient terminal 103. These data, include, but are not limited to: the patient measurement data; the data from the functional indicators; the qualifying data; the time/day of the patient measurements; the patient identification; and threshold values (or measures) for the patient. The server 101 also includes a memory 204 that stores messages from the patient and from the clinician. The memory 204 may be a read-only-memory (ROM) such as an electrically erasable programmable memory (EEPROM) or similar type of flash memory.

The server 101 also includes a text editor 205 in an example embodiment. The text editor 205 is implemented in known software and is adapted to receive the input from the interface of the clinician terminal 102 and to generate a text message based on the input.

The server 101 includes operating system (OS) software with application software written to perform tasks in accordance with the system 100 of the present teachings. In a specific embodiment, the OS software is ThreadX real time operating system (RTOS) software commercially available from Express Logic, Inc. San Diego, Calif. USA.

In operation, the server 101 receives data from the patient terminal 103. These data are stored in the database 203. The server transmits the data to the clinician terminal for analysis. Alternatively, the using algorithms provided in the application software, the server 101 can analyze the data and provide an appropriate message to the patient terminal.

After receiving the data, the clinician may send a personal message to the patient by inputting the message at the interface of the clinician terminal 102. The input message is transmitted to the server 101 and received at the text editor 205. The text editor 205 generates a message for transmission by the server 101 to the patient terminal 103.

In another example embodiment, audio messages are input at the interface of the clinician terminal. The server 101 includes known software adapted to convert voice messages into text data. These text data are provided to the text editor 205 for generation of a message to the patient. In another embodiment, the voice message is transmitted to the patient terminal 103 directly.

After the text editor 205 generates the message, the message is transmitted to the patient terminal 103 for the patient's use. Notably, the message may also be transmitted to a local memory 205. Illustratively, the stored message includes the author, the date and any acknowledgement received from the patient.

FIG. 3 is a simplified block diagram of the server 101 in accordance with another example embodiment. The server 101 includes many features described in conjunction with FIG. 2. In fact, the components and features of the server 101 presently described in connection with FIG. 3 may be incorporated into the server 101 described in connection with FIG. 2 thereby integrating the functions of both into one server.

The server 101 includes the interfaces 201, 202 adapted to effect the connections between the server 101 and the clinician terminal 102 and the patient terminal 103, respectively. The database 203 receives data from the patient terminal 103. These data include the device status data from the monitoring devices 105 and sensor devices 106. A device monitor engine 301 retrieves these data from the database 203 and algorithmically analyzes the data.

The algorithms of the present teachings are provided via application software (code) written from the OS of the server 101. The algorithms include comparisons to threshold values or patient data previously garnered and stored in the database 203.

Illustratively, the algorithms check: specific status data such as internal device state or operation sent by the functional indicators; the details factors of a specific measurement such as signal quality; time required to make a measurement; and patient actions or interventions during and around the time the measurement was made. The algorithms may also analyze the timing of different measurements to infer patient status or condition in addition to that indicated by the patient measurement.

The execution of the algorithms may be scheduled when a new measurement is communicated to the server 101 or may be scheduled periodically. The algorithms can then determine whether the patient, or the clinician, or both should be sent a message. If the patient can be reasonably expected to perform the recommended action and if medical intervention is not required, a message will likely be sent only to the patient terminal 103. The message sent, the time sent, and any patient acknowledgement from his terminal may be stored in the database 203.

The algorithms of the server 101 are also adapted to determine if intervention by the clinician may be needed. To this end, based on the data the algorithms may determine that the patient might need assistance, or further medical judgment. In addition, the algorithms may determine that the clinician needs to intervene with medical care. In any of these scenarios, a message may be sent to the clinician terminal 102. In an embodiment, the message sent, the time sent, and any patient acknowledgement from the patient terminal are stored in the database 203.

Based on the message received at the clinician terminal 102 from the server 101, the clinician can decide whether to send a message to the patient's terminal, or to otherwise intervene. Any message sent from the clinician, the time sent, and any patient acknowledgement may also be stored in the database.

In operation, if the device monitor engine 301 algorithmically determines that action must be taken, a command is sent to an action engine 302. The action engine 302 is also implemented in application software in the OS of the server 101. The action engine 302 generates a message of the type described previously described for transmission to the patient terminal 103. The message is transmitted via the interface 202. Optionally, the message is also transmitted to the clinician terminal 102 via the interface 201.

For purposes of illustration, suppose the data from the patient terminal includes device status or anomalous data that requires action. For example, suppose the data from the sensor 106 indicates that the battery on a device is at or below a threshold value. The algorithms implemented via the device monitor engine 301 of the server 101 compares the data with the threshold (e.g., minimum) battery voltage level stored in the database for the particular medical device 104. After the comparison, the action engine generates a message to check or replace the battery in the particular medical device.

In another illustration of the method, suppose a patient's weight is normally within a particular range over a recent period of time. These weight data are stored in the database 203 for this patient. If data from a number of measurements are received that are outside the range stored in the database, there may be a malfunction in the scale, or the scale may be in use by other than the patient. The algorithms of the device monitor engine 301 will compare the weight data received with the recent temporal weight range. In response, the action engine 302 may generate a message to check the accuracy of the scale and to remind the patient that other people should not use the scale. Thus, the source of the anomalous data may be determined and remedial action taken by the patient.

Continuing the present example, suppose that in response to the message, the patient confirms that the scale is functioning properly and that no other person has used the scale. If the patient has increased or decreased in weight beyond a threshold amount or other relative measure in a prescribed period of time, the algorithms may trigger messages to the clinician terminal 102 for action by the clinician in a manner consistent to that previously described.

In still other examples, self-tests and other monitoring may be effected. For example, suppose a patient is equipped with a pacemaker that includes a monitor device integrated into the pacemaker. Data from the monitor device can be transmitted to the patient terminal 103 and from the patient terminal 103 to the server 101. The data are provided to the database 203.

The device monitor engine 301 analyses these data algorithmically via application software written for these analyses. If, for example, the pacemaker is not functioning properly the action engine 302 can generate and transmit a message to the patient terminal instructing the patient to take appropriate action. Optionally, this message may be provided to the clinician terminal 102 by the server 101.

FIG. 4 is a flow-chart of a method in accordance with an example embodiment. The method incorporates the devices, components and algorithms of the example embodiments described in connection with FIGS. 1-3. As such, the method is best understood from a concurrent review of FIGS. 1-4. Moreover, common details of the devices, components and algorithms are not repeated so as to avoid obscuring the description of the illustrative method.

At step 401, data are gathered at the patient terminal 103 and are transmitted to the server 101. These data include measurement data from the medical devices 104 described previously. The server 101 stores these data in the database 203 for the particular patient. The server 101 also transmits the data to the clinician at step 402. After reviewing the data, at step 403 the clinician inputs a message at the clinician terminal 102 for the patient based on the data received. At step 404, the message is transmitted to the server 101 and to the patient terminal 103. Optionally, the patient may provide a message in reply to the message received at the terminal 103.

Beneficially, according to the method of the example embodiment, the clinician can provide feedback to the patient at the time of his/her choosing after reviewing the patient's recent information. As can be appreciated, the process is efficient for a number of reasons. For example, the clinician does not need to reach the patient by phone, and thus does not risk having to make multiple calls to the patient until reaching the patient. Furthermore, the time required to provide a message is comparatively small to the time of a dialog on the phone.

In addition, the measurements garnered may provide the clinician with a broader assessment of the patient's health status that likely would require in-person observations normally. For example, suppose the measurements gathered from a scale indicate that the patient steps off the scale numerous times in relatively close succession before an accurate reading is taken. Such data may be analyzed algorithmically at the server 101 or may be provided to the clinician by the server 101 for analysis. In the former instance, the server 101 may generate a message that the patient is unstable and may require attention. The same conclusion may be reached by the clinician. The action required may be as simple as providing a scale with handles.

In another illustration of the use and benefits of the present method, suppose the pulse oximeter has many aborted measurements because of poor perfusion. This may indicate compromised peripheral circulation. The server 101 may algorithmically determine the need for a message to the patient to warm his/her fingers before the measurement is made. Alternatively, the clinician may provide a similar message based on the data from the server 101.

FIG. 5 is a flow-chart of a method in accordance with an example embodiment. The method incorporates the devices, components and algorithms of the example embodiments described in connection with FIGS. 1-3. As such, the method is best understood from a concurrent review of FIGS. 1-3 and FIG. 5. Moreover, common details of the devices, components and algorithms are not repeated so as to avoid obscuring the description of the illustrative method.

At step 501 data are gathered for a medical device 104 by the monitor 105 or the sensor 106, or both. These data are provided to the patient terminal 103 via the communication link between the medical device 104 and the patient terminal. In an alternative embodiment, the data are transmitted directly to the server 101.

At step 502, the patient terminal 103 transmits the data to the server 101. The transmission of the data to the server 101 is via the communication link described previously.

At step 503, the data are stored at the database 203 of the server 101 and analyzed algorithmically by the device monitor engine 301. As described previously, in an illustrative embodiment these data may be compared to a threshold value by the device monitor engine 301. At step 504, from the analysis the device monitor engine 301 determines if action is required.

For example, suppose the data were from a self-test from the monitor of an EKG machine. If, when compared to acceptable values within calibration, the self-test data were within set limits, no action would be required because the EKG machine appears to be functioning properly. In this case, the method would return to step 501 and additional data is garnered repeating the process for the particular medical device 104.

However, if the self-test data were outside the accepted values, the method would continue at step 505. At this point, based on the analysis of the device monitor engine 301, the action engine 302 generates a message for transmission to the patient terminal. In the present illustration of the method, the message may instruct the patient to take a curative step such as contacting a service technician to repair the faulty device. At the completion of step 505, the process repeats at step 501. It is contemplated that the method of the present embodiment may be performed in parallel for all medical devices 104 of the system 100.

Beneficially, the functional indicators foster proper function of the medical devices 104 of the example embodiments. As can be appreciated, the patient's medical care can be more efficiently administered.

In view of this disclosure it is noted that the various methods and devices described herein can be implemented in hardware and software. Further, the various methods and parameters are included by way of example only and not in any limiting sense. In view of this disclosure, those skilled in the art can implement the present teachings in determining their own techniques and needed equipment to effect these techniques, while remaining within the scope of the appended claims.

1. An apparatus, comprising:
a patient terminal (103);
a medical device (104) adapted to garner measurements from a patient and to transmit data from the measurements;
a clinician terminal (102) adapted to receive manual inputs or audio inputs, or both; and
a server (101) adapted to receive the data and the inputs, wherein the server is operative to transfer the inputs to the patient terminal.

2. An apparatus as recited in claim 1, wherein the patient terminal (103) comprises a stationary device (107) and a remote device (108) adapted to communicate with the stationary device.

3. An apparatus as recited in claim 1, wherein the clinician terminal comprises a stationary device (109) and a remote device (110) adapted to communicate with the remote device (110).

4. An apparatus as recited in claim 1, wherein the server (101) further comprises a processor (200) and the processor (200) is adapted to convert the manual and the audio inputs into text and to transmit the text to the patient terminal (103).

5. An apparatus as recited in claim 1, wherein the medical device (104) is adapted to transmit the data to the patient terminal (103) and the patient terminal (103) is adapted to transmit the data to the server (101) or the medical device (104) is adapted to transmit the data to the server (101) without transmitting the data to the patient terminal (103).

6. (canceled)

7. An apparatus as recited in claim 1, wherein the server (101) is adapted to analyze the data and, based on the analysis, to transmit a message to the patient terminal.

8. An apparatus as recited in claim 1, wherein the data include a time and a date, or a signal quality, or both of each of the measurements.

9. A method, comprising:
measuring a vital sign;
transmitting data from the measuring to a server (101);
transmitting the data from the server to a clinician terminal (102);
based on the data, inputting a message to the clinician terminal (102); and
providing the message at the patient terminal (103).

10. A method as recited in claim 9, wherein the measuring vital sign further comprises measuring a time and a date of the measuring.

11. A method as recited in claim 10, wherein the inputting the message further comprises one of either:
reviewing the data and inputting a text message at the clinician terminal (102) or reviewing the data and inputting a voice message at the clinician terminal (102).

12. (canceled)

13. A method as recited in claim 9, wherein the transmitting to the server (101) further comprises one of either transmitting the data to the patient terminal (103) and transmitting the data from the patient terminal (103) to the server or not transmitting the data to the patient terminal (103).

14. (canceled)

15. A method as recited in claim 9, wherein the transmitting to the clinician terminal (102) further comprises transmitting the data to a stationary device (109) of the clinician terminal (102), and transmitting the data from the stationary device (109) to a remote device; and
the inputting further comprises inputting the message to the remote device (110) and transmitting the message to the stationary device (109).

16. An apparatus, comprising:
a patient terminal (103);
a medical device (104);
a functional indicator (105, 106) adapted to provide data on a status of the medical device (104); and
a server (101) adapted to receive the data, and based on the data to provide feedback to the patient terminal (103).

17. An apparatus as recited in claim 16, wherein the server (101) further comprises a processor (200) adapted to analyze the data and to provide the feedback to the patient terminal (103) based on the analysis.

18. An apparatus as recited in claim 16, wherein the functional indicator (105, 106) further comprises a sensor adapted to garner measurements from the medical device (104).

19. An apparatus as recited in claim 16, wherein the functional indicator (105, 106) further comprises a self-test circuit within the medical device (104) and the data are from a self-test routine performed with the self-test routine.

20. An apparatus as recited in claim 16, wherein the functional indicators (105, 106) are adapted to provide one or more of: data variance across a plurality of measurements from the medical device provided in a single reading; an elapsed time to acquire measurement data from the medical device; a time of day and a date of a measurement from the medical device.

21. A method, comprising:
gathering data from a functional indicator (105, 106) of a medical device (104);
transmitting the data from the functional indicator (104) to a server (101); and
based on the data, determining an appropriate action at the server (101).

22. A method as recited in claim 21, wherein the determining further comprises transmitting a message to a patient terminal (103), not transmitting a message to a patient terminal (103), or comparing the data to one or more measures, and based on the comparing performing the determining.

23. (canceled)

24. A method as recited in claim 21, further comprising, after the gathering, transmitting the data to a clinician terminal (103).

25. (canceled)

26. (canceled)

27. A method as recited in claim 21, wherein the data further comprise one or more of: a data variance across a plurality of measurements from the medical device provided in a single reading; an elapsed time to acquire measurement data from the medical device; a time of day and a date of a measurement from the medical device.
